(19) United States
(12) Patent Application Publication
(10) Pub. No.: US 2020/0015712 A1
(43) Pub. Date: Jan. 16, 2020

(54) Gathering and Analyzing Posture and Movement Data

(71) Applicant: ArthroKinetic Institute, LLC, Santa Barbara, CA (US)

(72) Inventor: Maury Hayashida, Santa Barbara, CA (US)

(21) Appl. No.: 16/035,398

(22) Filed: Jul. 13, 2018

(51) Int. Cl. A61B 5/11; H04W 4/38; G08B 21/04; G01P 15/18; A61B 5/00; G16H 40/63

(52) U.S. Cl. CPC A61B 5/1116; H04W 4/38; G08B 21/0446; G01P 15/18; A61B 5/0004; A61B 5/7246; G06F 3/147; A61B 5/0022; A61B 5/6823; A61B 5/746; A61B 5/486; G16H 40/63; A61B 5/742

(57) ABSTRACT

A method includes: receiving, by a first processing device, first data from a first inertial sensor positioned on a user, generated when the user is in a body position; analyzing the first data to identify a posture of the body position; receiving, by the first processing device, second data from a second inertial sensor positioned on the user, wherein the second data are generated when the user performs a fundamental movement; analyzing the second data to identify a distinguishing characteristic of the fundamental movement; determining whether a correlation exists between the posture of the body position and the distinguishing characteristic of the fundamental movement; and, if a correlation between the posture of the body position and the distinguishing characteristic of the fundamental movement is determined to exist, displaying an indication of the correlation to the user on a second processing device.

### CROSS REFERENCE TO RELATED APPLICATION

This application is related to co-pending U.S. application Ser. No. 15/724,099 filed on Oct. 3, 2017, which is hereby incorporated by reference, as if set forth in full in this specification.

### FIELD OF THE DISCLOSURE

Various embodiments described herein relate generally to the field of analyzing data characterizing user posture and user movement, and in particular to methods and systems that facilitate such data generation and analysis using wearable inertial measurement unit (IMU) sensors and data processing to yield diagnostically useful indications of posture quality, movement quality, and correlations between posture and subsequent movement.

### BACKGROUND

Sustained static body positions & repeated movements may be the primary cause of neuromusculoskeletal injuries or conditions. Holding poor quality postures (such as tending to lean over to one side or the other) in those body positions before carrying out particular movements may be particularly problematic in this regard. Holding certain body position postures may even have a positive impact on some subsequent movements.

It may therefore be very beneficial to determine whether such correlations exist for an individual user, so that, for example, the user may be guided to adopt better postures, or to change body positions more frequently, before carrying out particular movements.

### BRIEF DESCRIPTION OF THE DRAWINGS

FIG. 1A illustrates an operating environment including connected sensor and processing devices according to some embodiments of the present invention.

FIG. 1B illustrates an operating environment including connected sensor and processing devices according to some other embodiments of the present invention.

FIG. 2 shows a functional block diagram of a sensor device according to some embodiments of the present invention.

FIG. 3 shows a functional block diagram of a processing device according to some embodiments of the present invention.

FIG. 4 is a flowchart of a method according to some embodiments of the present invention.

FIG. 5 is a flowchart of a method according to some embodiments of the present invention.

FIG. 6 is a flowchart of a method according to some embodiments of the present invention.

### DETAILED DESCRIPTION

### I. Introductory Overview

Various systems, devices, and methods are directed to the unique placement of an inertial measurement unit (IMU) sensor in/around the center of mass of a user, the sensor providing data that may be analyzed by a processing device to: (1) identify a posture (e.g. slouching, leaning to the left/right, crossing legs, etc.) of the user's body position (e.g. sitting, standing, lying down, etc.); (2) identify a characteristic (e.g., limp, stagger, etc.) of the user's movement (running, jumping, etc.); and (3) if a correlation between the posture and the movement characteristic is determined to exist, provide an indication of that correlation, and, if necessary, instigate corrective action. In some cases, one or more additional IMU sensors may be used to provide some of the posture and movement data gathered from the user and analyzed.

For example, some ways to characterize sitting posture include sitting crossed legged in a closed position (right over left, left over right), sitting crossed legged in an open position (right over left, left over right), sitting leaning (left, right), and so on. Some ways to characterize standing posture include, for example, leaning (left, right). Some ways to characterize lying down include, for example, lying on one side (left, right), lying prone, or lying supine with a leg turned out on one side while not on the other.

Establishing whether such correlations exist for an individual user may be useful for (1) pre/during/post measurement for clinical decision making, (2) biofeedback (patient learning; post symptoms or no symptoms; predisposition or perpetuation of a problem), (3) insurance documentation of progress or lack-of-progress (4) identifying injury risk by perpetuating postural or movement combinations that are asymmetrically dominant or dysfunctional and (5) identifying patterns/habits that limit movement performance and functional outcomes of the user.

### II. Example Operating Environment

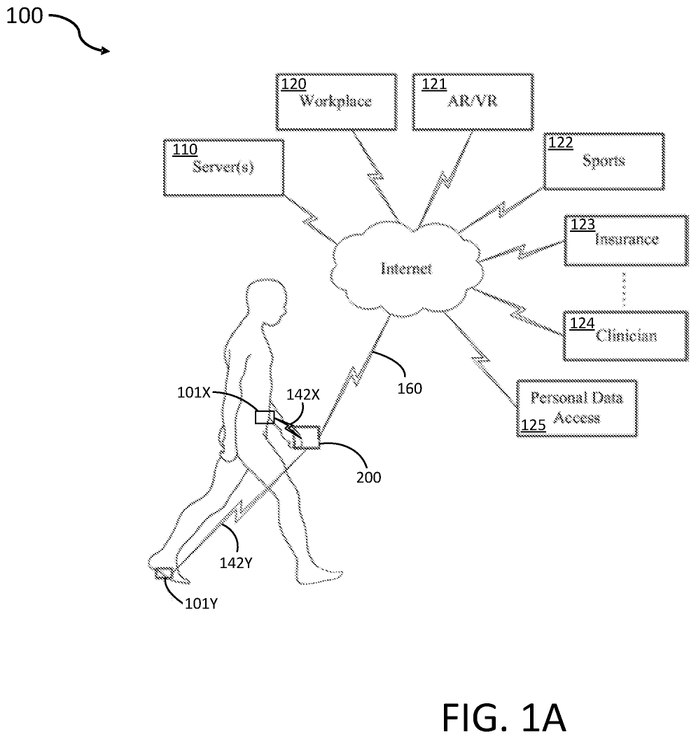

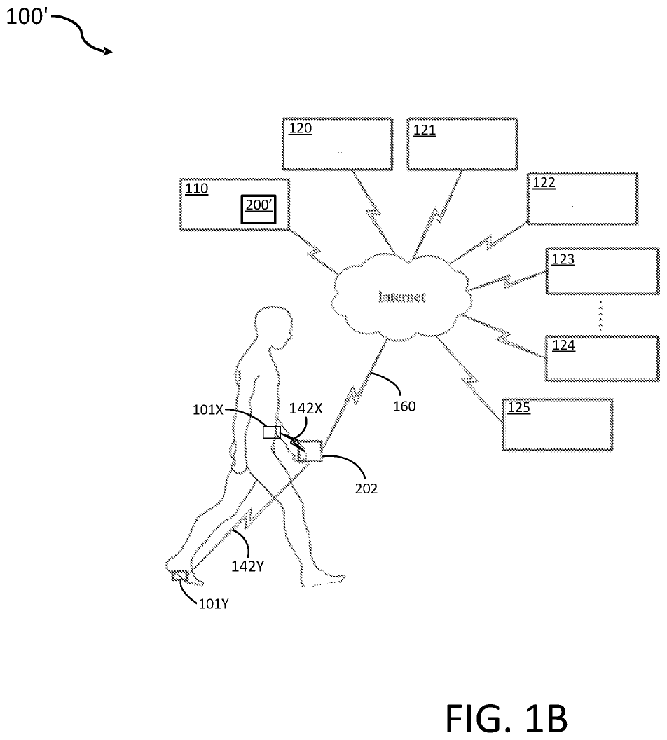

FIG. 1A shows an example configuration of an inertial sensor system 100 in which one or more embodiments disclosed herein may be practiced or implemented. Inertial sensor system 100 includes a user wearing two inertial sensor devices 101X and 101Y (though in some other embodiments, not shown, the user may wear more than two sensor devices, or just one) and a processing device 200 that may be held in the user's hand or worn, for example on the user's wrist or another part of their body. The sensor devices 101X and 101Y are shown transmitting data through wireless channels 142X and 142Y, respectively, to processing device 200. In general, each sensor device may generate data indicative of both position and movement, but according to the location of the sensor, the data generated may be more strongly representative of either position or movement or certain characteristics thereof. In a “single sensor” embodiment, the single sensor, typically positioned near the user's center of mass, may provide data indicative not only of position (and posture thereof) but also movement (and characteristics thereof).

Additionally, processing device 200 is shown communicating through wireless channel 160 via a network (e.g., a local area network or LAN, cellular network, and/or the Internet) to one or more servers 110 or other computing devices 120-125 that are accessible by entities authorized by the user.

In the embodiment shown in FIG. 1A, an artificial intelligence (AI) agent within processing device 200 carries out an analysis of the sensor data, and communicates back to the user as required. Wireless channel 160 may be used to transmit data and/or communication information to other devices 110 and 120-125, for example, for data storage, further data processing, data display, etc.

FIG. 1B shows an alternative example configuration in other embodiments of an inertial sensor system 100′, in which processing device 200′, including an AI agent, is situated remotely from the user, either at network server 110 as shown, or, in other cases, at another computing device 120-125. Processing device 200′ is fed data originally generated by the sensor devices 101X, 101Y through an intermediary processing device 202 that is positioned on or close to the user. Processing device 200′ then performs the core data analysis and communicates back to processing device 202 or other devices (110, 120-125) across the network as needed. In this example, processing device 202 may act as an intermediary device sending data between the sensor device(s) 101X, 101Y and the processing device 200′, or it may do some data processing prior to sending data between the sensor device(s) 101X, 101Y and the processing device 200′.

In the embodiments of FIGS. 1A and 1B, IMU device 101X is placed at, near, or around the waist of the user, where it can be approximated to be at the center of mass of the user. It may, for example, be attached to or embedded within a belt or other garment, or attached to the skin with an adhesive patch. In these embodiments, a second IMU sensor device 101Y may be placed in or around a second part of the body. For example, sensor device 101Y may be placed in the sole of a shoe or in an orthotic shoe insole (also called an “orthotic shoe insert”) placed within the shoe, on or around the knee, on or around the shoulder, arm, or elbow, or other places on the body. In some embodiments, devices 101X and 101Y include SweetSpot™ sensor technology, as described, for example, in U.S. patent application Ser. No. 15/724,099, referenced above.

### III. Example Sensor Devices

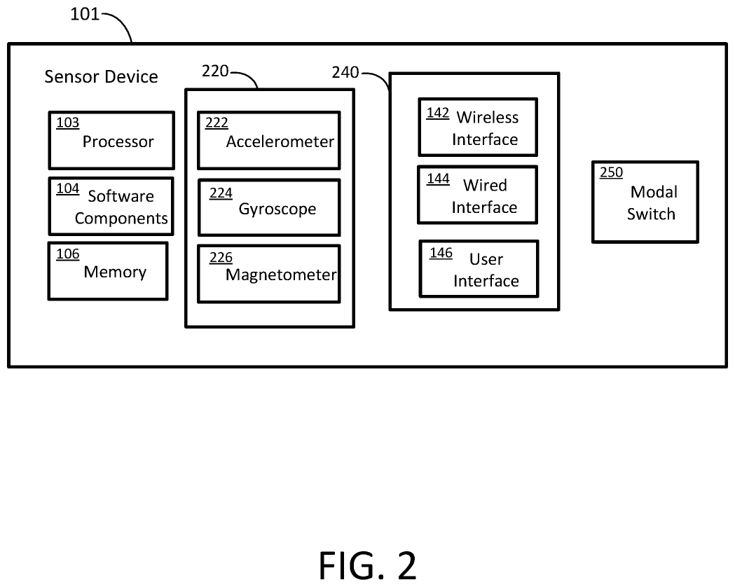

In some embodiments, sensor devices 101X and 101Y provide 3-axis data with one or more associated timestamps. FIG. 2 shows a functional block diagram of an example inertial sensor device 101 according to one embodiment. Sensor device 101 includes one or more processors 103, software components 104, memory 106, a motion detection sensor block 220, a data interface 240, and a modal switch 250. Motion detection sensor block 220 may include one or more combinations of distinct types of inertial sensors such as an accelerometer 222, a gyrometer (used herein interchangeably with “gyroscope”) 224, and a magnetometer 226. Data interface 240 may include one or both wireless (142) and wired (144) interfaces, and additionally may include user interface 146.

Processor 103 may include one or more general-purpose and/or special-purpose processors and/or microprocessors that are configured to perform various operations of a computing device. Memory 106 may include a non-transitory computer-readable medium configured to store instructions executable by the one or more processors 103. For instance, memory 106 may be data storage that can be loaded with one or more of the software components 104, executable by the one or more processors 103 to achieve certain functions. In one example, the functions may involve collecting inertial data from the one or more sensors 222-226 and transmitting the inertial data to another device over the data interface 240.

As noted above, the motion detection sensor block 220 includes one or more inertial sensor components such as accelerometers, gyrometers, and magnetometers. Considering a single axis for simplicity, an accelerometer essentially measures linear acceleration along that axis, from which force can be derived, a gyrometer measures angular velocity about that axis, from which rotational motion direction can be derived, and a magnetometer measures magnetic flux density along that axis, from which orientation with respect to the earth's surface can be derived. Each sensor 222, 224, and 226 may have multi-axis (typically 3 axis) sensing capability, and each sensor may be able to collect sensor data simultaneously or substantially simultaneously. For example, the accelerometer 222 may be able to collect acceleration force data at the same time the gyrometer 224 collects rotational motion data. Similarly, the magnetometer 226 may be able to collect orientation data at the same time the accelerometer 222 collects acceleration force data. Other combinations exist.

The data interface may be configured to facilitate a data flow between the sensor device 101 and one or more other devices, including but not limited to data to/from other sensor devices or processing devices 200 (to be described below). As shown, the data interface 240 may include wireless interface(s) 142 and wired interface(s) 144. The wireless interface(s) 142 may provide data interface functions for the sensor device 101 to wirelessly communicate with other devices (e.g., other sensor device(s), processing device(s), etc.) in accordance with a communication protocol (e.g., any wireless standard including IEEE 802.15, 802.11a, 802.11b, 802.11g, 802.11n, 802.11ac, 4G or 5G mobile communication standard, and so on). The wired interface(s) 144 may provide data interface functions for the sensor device 101 to communicate over a wired connection with other devices in accordance with a communication protocol (e.g., USB 2.0, 3.x, micro-USB, Lightning by Apple, IEEE 802.3, etc.). While the data interface shown in FIG. 2 includes both wireless interface(s) 142 and wired interface(s) 144, data interface 240 may in some embodiments include only wireless interface(s) 142 or only wired interface(s) 144.

User interface 146 may generally facilitate user interaction with the sensor device 101. Specifically, user interface 146 may be configured to detect user inputs and/or provide feedback to a user, such as audio, visual, audiovisual, and/or tactile feedback. As such, user interface 146 may include one or more input interfaces, such as mechanical buttons, “soft” buttons, dials, touch-screens, etc. In example implementations, user interface 146 may include a modal switch 250, configured to toggle the operation of the sensor device 101 between operating modes. For example, some example modes may include programming mode, diagnostic mode, and operational mode. Other examples are also possible.

In some embodiments, sensor device 101X and/or 101Y may be accessible by the user to manually turn the sensor device on or off, while in other cases the operation of the sensor device may include a “sleep mode” where the sensor device remains in a dormant (e.g., low-power) state but is automatically turned to a full “on” state by motion. In some embodiments, the sensor device may turn off or return to sleep mode after a predetermined time interval, if no motion is detected during that interval.

In some embodiments, sensor device 101X and/or 101Y may be accessible by the user so that, for example, a battery may be replaced, or so that the entire sensor device may be replaced by another sensor device. In some embodiments, the battery may not be accessible and the insole or insert would be discarded once the battery is depleted. Other examples may readily be envisaged.

The embodiments of FIGS. 1A and 1B involve the positioning of sensor device 101X on or near the center of mass of the user, typically against or around the waist or pelvis, where data can be readily generated indicative of body position and posture. In these embodiments, sensor device 101Y may be positioned in an insole or shoe insert, delivering data directly indicative of foot and ankle movement, but the data provided by such sensor devices may also be used to infer facts about movement at other joints of the body, such as, for example, the knee, hip or pelvis. Other embodiments may involve similar sensor devices positioned on or near other parts of the body, such as the knee, hip, or pelvis, so that data gathered and delivered to the processing device may be more directly indicative of the biomechanics of that body part, and interpreted in terms of corresponding fundamental movements.

In some embodiments, the analysis of data by the processing device includes comparing one or more features of the measured data with features of pre-established “signatures.” The pre-established “signatures”, obtained either from the same user or one or more other users performing the same movement or holding the same body position and posture, may be stored on the local processing device, on a server in the cloud, in a local network server, or a combination of these. The comparison process may include, for example, using data thresholds and/or using machine learning to identify features. Based on the analysis, the device may provide, for example, (1) an indication of the fundamental movement that the user is performing or has performed (e.g., walking, running, and so on), (2) an indication whether the user wearing the sensor 101 is moving incorrectly (or correctly, if so desired); and/or (3) an indication that the sensor 101 itself needs adjustment. Methods by which “signatures” may be established are described in patent application Ser. No. 15/724,099, referenced above.

Referring back to FIGS. 1A and 1B, some additional aspects of the data collection and communication will now be described. In the embodiments of FIG. 1A, each sensor device (e.g. 101X) independently transmits data over the corresponding wireless data interface (e.g. 142X) to processing device 200 using wireless technology such as, for example, Wi-Fi®, Bluetooth®, Bluetooth Low Energy (BTLE), NFC, etc. Communication may be direct (point-to-point) between each sensor device and the processing device 200 or communication may be routed through an Access Point or another networking relay device that can be used to propagate data packets from one networking device to another. The data may be sent periodically during a movement/exercise or when a movement/exercise is complete. The data transmission may also be contingent upon having a good network connection and/or battery life. For example, the data may be sent upon detection of the completion of one or more movement/exercises among a series of movements/exercises (i.e. after one or more laps, miles, reps, etc. are detected) or periodically at regular time intervals (e.g., 1, 5, 10 sec).

In the embodiments of FIG. 1B, the sensor data are transmitted wirelessly to the intermediary processing device 202 (e.g., mobile phone, tablet, PC/Mac, connected watch, glasses or other connected wearable device) that may do some preliminary processing before sending the partially processed data on to processing device 200 across the network. The communication between the intermediary processing device 202 and the server 110 or other computing device 120-125 may use wired or wireless technologies and typical networking protocols.

Throughout this disclosure, the term “fundamental movement” is defined to be any one of the following movements (or substantially similar movements): walking, running, single-leg jumping, double-leg jumping, skip and hop, squatting, partial squatting, shuffle direction change, lunge, throw, and catch. Each of these fundamental movements may be considered to include one or more phases, which in turn may be considered to include one or more sub-phases.

### IV. Example Processing Devices

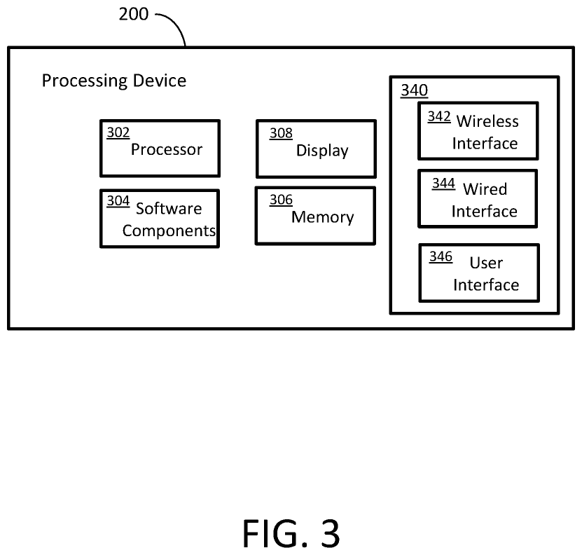

FIG. 3 shows a functional block diagram of an example processing device 200 according to one embodiment of the present invention. Processing device 200 includes one or more processors 302, software components 304, memory 306, a display 308, a data interface 340 which may include a wireless interface 342 and/or a wired interface 344, and a user interface 346. The processing device may be a device where application software may be installed, such as for example, a mobile phone, tablet, PC/Mac, connected watch, smart glasses, or other connected wearable device.

Processor 302 may include one or more general-purpose and/or special-purpose processors and/or microprocessors that are configured to perform various operations of a computing device. Memory 306 may include a non-transitory computer-readable medium configured to store instructions executable by the one or more processors 302. For instance, memory 306 may be data storage that can be loaded with one or more of the software components 304, executable by the one or more processors 302 to achieve certain functions.

The data interface 340 may be configured to facilitate a data flow between the processing device 200 and one or more other devices, including but not limited to data to/from the sensor device 101 or other networked devices. The data interface 340 may include wireless interface(s) 342 and wired interface(s) 344. Wireless interface(s) 342 may provide data interface functions for the processing device 200 to wirelessly communicate with other devices (e.g., other sensor device(s), processing device(s), etc.) in accordance with a communication protocol (e.g., any wireless standard including IEEE 802.15, 802.11a, 802.11b, 802.11g, 802.11n, 802.11ac, 4G and 5G mobile communication standard, and so on). The wired interface(s) 344 may provide data interface functions for the processing device 200 to communicate over a wired connection with other devices in accordance with a communication protocol (e.g., USB 2.0, 3.x, micro-USB, Lightning by Apple, IEEE 802.3, etc.). While the data interface shown in FIG. 3 includes both wireless interface(s) 342 and wired interface(s) 344, data interface 340 may in some embodiments include only wireless interface(s) 342 or only wired interface(s) 344.

User interface 346 may generally facilitate user interaction with processing device 200 and control of sensor devices (101X, 101Y). Specifically, user interface 346 may be configured to detect user inputs and/or provide feedback to a user, such as audio, visual, audiovisual, and/or tactile feedback. As such, user interface 346 may include one or more input interfaces, such as mechanical buttons, “soft” buttons, dials, touch-screens, etc. In example implementations, user interface 346 may take the form of a graphical user interface configured with input and output capabilities. Other examples are also possible.

Display 308 may generally facilitate the display of information. For example, some results of the analysis, notifications, suggested movement corrections, etc. may be displayed to the user via display 308.

### V. Example Embodiments

Example embodiments of the present invention will be described below in terms of three main categories: A) determining correlations between body position/posture and movement quality; B) limiting data transfer between elements of the system according to preestablished thresholds; C) memorializing movement data associated with significant events. Throughout this disclosure, when a processing device is described as analyzing data, identifying features, determining correlations, etc. it should be understood that an AI agent within the processing device may be carrying out the described analysis, identification, determination, etc.

### A) Determining Correlations

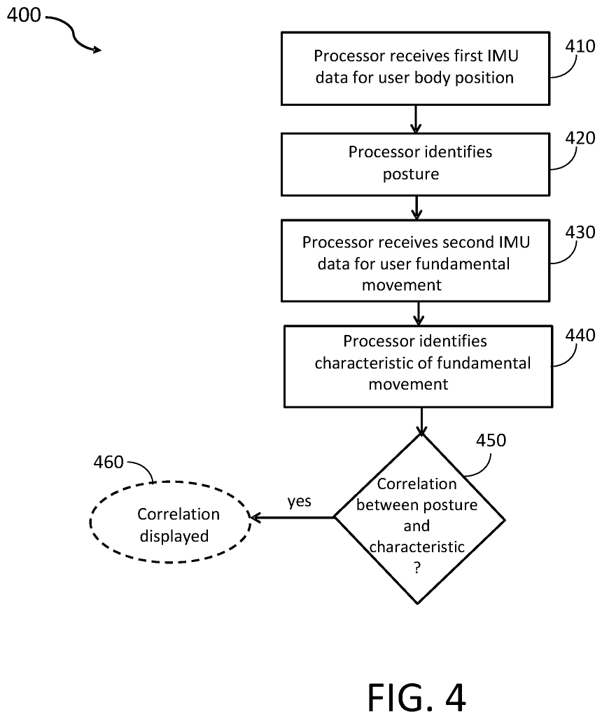

Method 400 in FIG. 4 shows an embodiment of an example method that can be implemented within an operating environment such as that shown in FIG. 1A, including IMU sensors 101X, 101Y, and processing device 200 in system 100, or such as that shown in FIG. 1B including IMU sensors 101X, 101Y and processing devices 200′ and 202 in system 100′. Method 400 is directed towards the determination of correlation between postures (and/or duration) of body position and characteristics of fundamental movement performed by the user.

At step 410, a first processing device (e.g. 200, 200′) receives first data sent over a wireless communication channel from a first IMU sensor positioned on a user, the data being recognized by the processing device as indicating a known body position such as standing, sitting, kneeling, lying down, etc. The data will typically comprise 3 to 6-axis IMU data (although in some cases the data may comprise anywhere in the range from 1-axis or 2-axis up to 9-axis data) and at least one associated timestamp.

At step 420, the first processing device analyzes the first data to identify a posture of the body position that was recognized at step 410. In some embodiments, this analysis involves comparing the data to a first pattern, representative of the posture, to find a match, within a first tolerance, between a portion of the first data and the first pattern. For example, the processing device may have access to stored patterns representative of leaning to the left, leaning to the right, tipping forwards, and tipping backwards, for a seated body position. Then, when the processor receives data indicative of a seated position, the processor may compare the data to those stored patterns and find a match between some portion of the data and a pattern indicating a “lean to the right” posture. The stored patterns may have been gathered from one or more subjects. In one embodiment, the stored patterns may have been gathered from the same individual user from whom the first data is being gathered.

At step 430, the first processing device receives second data sent over a wireless communication channel from a second IMU sensor positioned on the user, the second data being recognized by the processing device as indicating a known fundamental movement, such as walking, running, jumping etc. (In some embodiments, as noted above, only one sensor is used, meaning that the first sensor is the same sensor as the second sensor.) The data will typically comprise 3-6 axis IMU data (although in some cases anywhere in the range from 1-axis or 2-axis up to 9-axis data) and at least one associated timestamp.

At step 440, the first processing device analyzes the second data to identify a distinguishing characteristic of the fundamental movement. In some embodiments, this analysis involves comparing the second data to a second pattern, representative of the distinguishing characteristic, to find a match, within a first tolerance, between a portion of the second data and the second pattern. For example, after the second data was recognized at step 430 as indicating a running movement, analysis at step 440 may show that part of the data matches a pattern of a turning tendency in the left ankle.

At step 450, the first processing device determines whether a correlation exists between the posture of the body position and the distinguishing characteristic of the fundamental movement. In some embodiments, this determination is carried out with the help of the received timestamp data, by first determining a first time at which the user is in the posture of the body position; next, determining a second time at which the user performs the distinguishing characteristic of the fundamental movement; and then, evaluating the time interval between the first time and the second time as a possible first indicator of the correlation. For example, it may be found that standing with a pronounced lean to one side seems to affect a subsequent single-leg jumping movement, if that movement occurs with 15 minutes of being in that standing posture, whereas, if over 24 hours have elapsed between the pronounced lean and single-leg jumping movement, a correlation may not be taken as being very likely.

Another common example occurs if a user sits in a right ‘open leg crossing’ for either a sustained period of time (designated, for example, as 2 minutes or longer) then begins to walk immediately following that posture, and a step length reduction on the left leg stepping forward is identified. This may amount to a positive correlation, indicating cause and effect. This walking characteristic may be recognized as permanent or transitional/temporary and over time may be seen periodically, as related to sitting in that way.

In some other embodiments, the step 450 determination is carried out, again with the help of the received timestamp data, by first determining a first duration time for which the user remains in the posture of the body position, and then evaluating the first duration time as a possible second indicator of the correlation. For example, it may be found that sitting cross legged for 30 minutes with a forward slouch correlates with limping or, more specifically, decreased step length bilaterally and/or increased lateral sway in a subsequent walking movement, whereas sitting in that posture for just 10 minutes might have no observable effect.

In yet other embodiments, the step 450 determination is carried out, again with the help of the received timestamp data, by first determining a second duration time for which the user remains performing the distinguishing characteristic in the fundamental movement; and then evaluating the second duration time as a possible third indicator of the correlation, and as a measure of long term significance of the correlation. For example, it may be found that standing dominantly to one leg, say the right leg, where weight is shifted predominantly to the right, affects the smoothness of a subsequent running movement in the form of a ‘pelvic drop’ on the left when in the right legged weight bearing/stance phase of the run, but that the effect is a transient one, only obvious in the first few minutes of the run. Or, in another example, it could be found that the effect becomes more pronounced over time, and/or persists as a chronic condition, whenever the user runs for more than about 20 minutes. The latter situation is obviously a more serious one that may warrant greater consideration. Indeed, a temporary prevalence that becomes permanent or a progressively dominant feature is significant and worth gathering data on, in hopefully helping to prevent a developing dysfunction that leads to injury or reduced performance

At step 460, if a correlation between the posture of the body position and the distinguishing characteristic of the fundamental movement has been determined (at step 450) to exist, a second processing device worn by or positioned in close proximity to the user displays an indication of the correlation. In embodiments where processing device 200 is at a remote server or other computing device, and processing device 202 is present in close proximity to the user, first processing device 200 transmits instructions to second processing device 202 to display the indication. In embodiments where processing device 200 is in close proximity to the user and processing device 202 is absent or not in use, of course no long distance transmission of instructions is necessary for the required display to occur.

Optionally, the result obtained at step 450 may also be stored at the first and/or second processing device, or at another computing device within the networked system. Similarly, the result may be displayed at any one or more of these computing devices.

Returning to step 420, in some embodiments, after the first processing device identifies a posture of the body position, the device may evaluate the posture for quality or correctness. For example, if the first data are indicative of a forward stooped sitting posture, but the stoop is a slight one, and only held for a short time, the posture may be deemed as being of acceptable quality, but if the stoop is more pronounced and/or held for a longer time, an alert may be generated and displayed to the user by the processing device. The alert may be, for example, a visual one, such as an indication displayed on a screen of the processing device, or it may be an audible or tactile notification provided by the processing device. Other examples exist. The determination of what is acceptable or correct may be made on the basis of comparison of one or more portions of the first data to stored patterns of position data, generated by the same individual user or by one or more other users.

Similarly, at step 440, after the the first processing device identifies a distinguishing characteristic of the fundamental movement, the device may evaluate the characteristic for quality or correctness. For example, if the second data are indicative of a tendency towards an ankle turn while running, that is deemed risky, an alert noting the problem may be generated and displayed to the user by the processing device. The alert may be, for example, a visual one, such as an indication displayed on a screen of the processing device, or it may be an audible or tactile notification provided by the processing device. The determination of what is problematic as opposed to acceptable may be made on the basis of comparison of one or more portions of the first data to stored patterns of fundamental movement data, generated by the same individual user or by one or more other users.

### B) Limiting Data Transfer

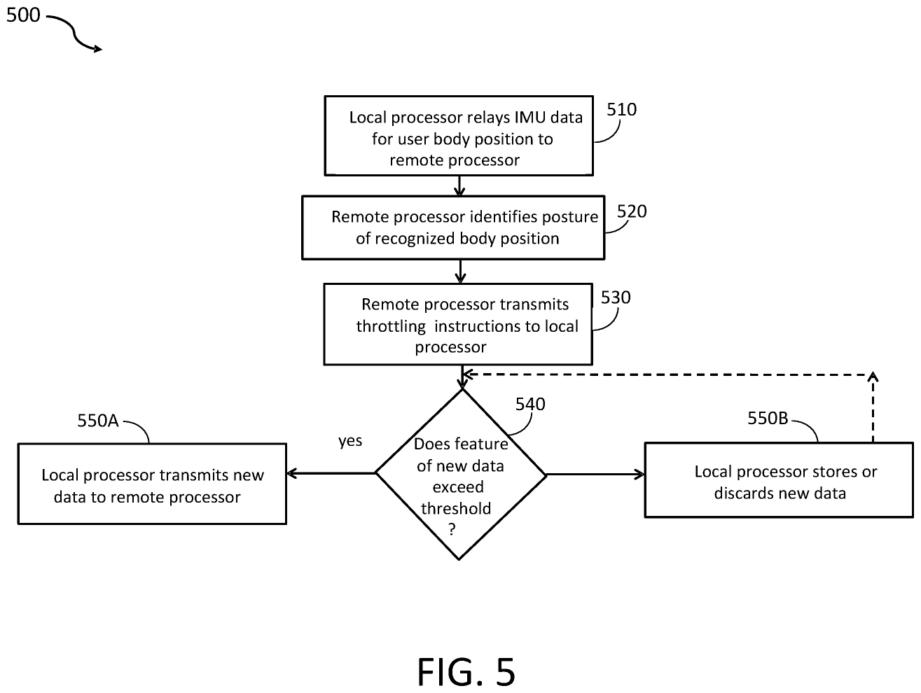

Method 500 in FIG. 5 shows an embodiment of an example method that can be implemented within an operating environment such as that shown in FIG. 1B, including sensors 101X, 101Y, and processing devices 200′ and 202 in system 100′. Method 500 is directed towards limiting data transfer between the sensors and the processing devices, and between the processing devices, so that, after the initial identification of a particular posture, only data that relate to features of that posture of particular interest (for examples, significant changes) are sent over the network to be analyzed at the remote processor. Similar methods, not shown, may be applied to limit data transfer during the identification of a particular posture, a particular movement, and/or features of that posture or movement.

At step 510, a local processing device (e.g. 202) worn by or in close proximity to a user, receives data sent over a wireless communication channel from a first IMU sensor positioned on a user and transmits the first data across a network to a remote processing device (200′) at a server or other computing device (110, 120-125). As noted above, the data will typically, but not always, comprise 3-6 axis IMU data and at least one associated timestamp.

At step 520, the remote processing device identifies a body posture of the position that caused the data to be generated at the first IMU sensor. At step 530, the remote processing device transmits instructions, determined by the posture and position, back to the local processing device.

At step 540, the local processing device analyzes new data received from the sensor by comparing a feature of the data with a threshold value for the identified posture and position. The threshold value may have been included in the instructions sent at step 530 from the remote processing device. The local processing device transmits the new data on to the remote processing device for further analysis if and only if the threshold is exceeded. Step 550A shows this conditional transmission. If the threshold is not exceeded, the method proceeds to step 550B, where the new data is either stored without further processing, or discarded. Step 540 therefore uses the threshold value to determine whether or not new data will be transmitted to the remote processing device.

A similar method (not shown) may be carried out to limit data transfer where the generated data are indicative of fundamental movements rather than static body positions. Instead of identifying posture of the body position at step 520, the distinguishing characteristic of the fundamental movement would be identified. In both cases, the methods serve to limit the transmission demands on elements of the system, so that data containing no indications of special interest—data generated during a long walk where no particular problems arise, for example—is either stored locally or simply discarded without using significant computational and transmission resources.

In some embodiments (not shown), after step 530, instead of the local processing device carrying out the comparison at step 540, the local processing device may relay the instructions from the remote processing device to the sensor device, in effect instructing the sensor device to cease transmitting data to the local processing device unless the sensor device itself determines that a feature of the generated data exceeds a threshold value for the identified position and posture (or fundamental movement and distinguishing characteristic). This scenario, requiring the sensor device to carry out step 540, clearly requires more intelligence at the sensor device than the alternatives described above, but may achieve even greater reduction in data transmission demands of the wireless communication channels.

The thresholds used in the methods described above may be derived from position and movement data previously gathered from IMU sensors used by the same user or one or more other users.

### C) Memorializing Movement Data

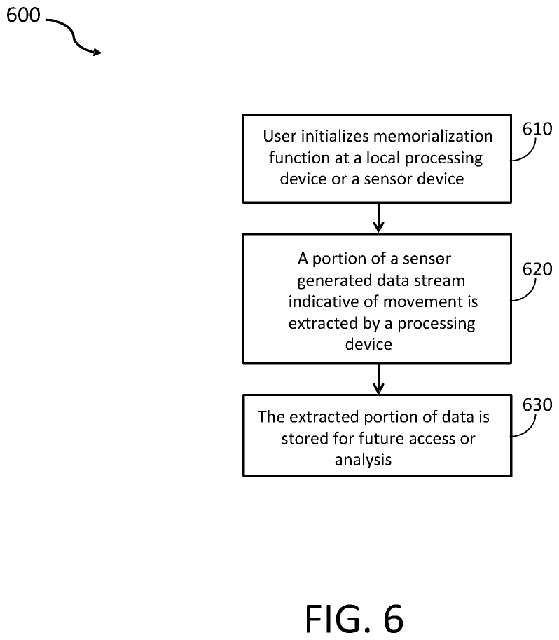

Method 600 in FIG. 6 shows an embodiment of an example method that may be implemented within an operating environment such as that shown in FIG. 1A, including IMU sensor device(s) (e.g., 101X, 101Y), and processing device 200 in system 100, or such as that shown in FIG. 1B including IMU sensor device(s) (e.g., 101X, 101Y) and processing devices 200′ and 202 in system 100′. Method 600 is directed towards the storage of data generated during the performance of a fundamental movement.

At step 610, the user initiates a memorialization function. In some embodiments, this initialization occurs at the user interface of a processing device worn by or in close proximity to the user (interface 346 of processing device 200 in the case of FIG. 1A, or a corresponding user interface on processing device 201 in the case of FIG. 1B) In some other embodiments, a sensor device such as 101X that is worn on the wrist or on or around the waist (or is in some other way conveniently accessible to the user) may include its own user interface, at which the memorialization function may be initiated.

A typical means of initialization may be pressing a button on the processing or sensor device, possibly with a pattern of presses that causes different types of memorialization to be initiated, as described further below.

At step 620, a processing device, which may be one worn by or in close proximity to the user (e.g., device 200 in FIG. 1A or 201 in FIG. 1B), or one situated remotely (e.g., device 200′ in FIG. 1B), wirelessly receives a stream of movement data generated by a sensor device and extracts a portion of the data according to the type of memorialization initialized. In cases where a sensor device user interface was used rather than a processing device user interface, the sensor device doing the receiving and extraction could be a different sensor device than the one at which the user initiated memorialization. In other cases where a local processing device user interface was used to initiate memorialization, the processing device receiving the data may be the same local processing device or a different processing device.

At step 630, the extracted portion of data is stored for later access and analysis as desired. Storage may be local or remote, or a combination of the two.

Returning to step 610, two main types of memorialization may be envisaged. In the first type, looking forward in time, a user may want a particular movement or sequence of movements that he or she is about to undertake to be recorded. A skateboarder, for example, may feel confident that the next half-pipe trick will be an especially good one, and so may want the data corresponding to body movements during that trick to be stored, and made available for future reference and study. A pattern of button presses may code the necessary instructions—maybe one long press followed by two short ones would signify to the processor that data corresponding to a time beginning immediately after the button presses and ending 2 minutes later are the data of interest, so (at step 620) the processor would extract data generated by any movement sensor on the user during that 2-minute time interval and (at step 630) arrange for those extracted data to be stored.

In another embodiment of the “looking forward in time” type, the user may not want elapsed time to be the sole determinant of which data should be stored, but for the significance of subsequent movements to be considered. For example, a particular pattern of button presses may instruct the processor to consider all data received for a certain time (say 30 minutes) after the presses, but to only extract data that the processor recognizes as indicative of movements or movement characteristics of particular interest. This might be helpful if an athlete, for example, has to wait for an imprecisely determinable time until a certain piece of equipment is available, but then needs to be ready to act fast and carry out a desired movement without pausing to interact with the processing device. It might also be helpful if the user expects to make several attempts to perform some movement, and only wants those attempts that are successful to be memorialized.

In another type of memorialization, looking back in time, a user may want data that was previously generated and transmitted to a processing device to receive particular attention. The hypothetical skateboarder, for example, may only realize after completing a trick that that trick seemed perfect. In another example, a user may be a runner who twists an ankle, and wants a record of the previous 30 minutes of activity to help identify possible causes, warning indications etc.

In such “looking back” cases, a different pattern of button presses (or other means of communicating with the user interface) may code for a different set of instructions—maybe a double tap would signify that data received in the past rather than the future is of interest, and the user might then immediately follow up by providing input on how far back in time the data are of interest, and for what duration, that input itself being coded in terms of number and duration of button presses. In other cases, the processing device may react to the double tap (or other indication that memorialization of past activity is desired) by asking the user via a display screen or audio interface for the necessary input on timing, on which to base the data extraction.

### VI. Use Scenarios

In this section, several specific use scenarios are presented to illustrate how embodiments described above might be used in practice.

### A) Athletic

A professional basketball player wears a sensor device (e.g., 101X in FIGS. 1A and 1B) at waistline between games during waking hours (avg. 3-4 days; 12 hr. intervals). During onboarding of the device 101X, the player provided information, for example, on user goals [e.g., multiple choice with categories of injury risk reduction, performance development, sport specific, health/wellness], notifications [e.g., push notifications, real-time, which devices, etc.], and information sharing [e.g., to his coaches, medical staff, etc.]. For example, the player may be interested to:
1) understand reason for recurrent low back pain/stiffness
2) learn if there's anything making him ‘injury prone’
3) learn how to be efficient in ‘transition’ on the court.

Because the player identified one of his goals was increasing performance with court ‘transition’, the processing device (e.g., 200, 202 in FIGS. 1A and 1B) prompts him to record a 5 sec. court run straight ahead as well as straight run while dribbling ball as part of a customization option during onboarding. [This may serve as baseline data for comparison].

Because there is a bit of an ‘educational curve’ regarding the sensor device 101X, the processing device 200, 202 displays a brief description of why it's important for him to wear the sensor device 101X during non-workout times (minimum 3-4 days; 12 waking hours) in order to track real-life postural and movement behaviors that impact the mind & body when translated to sport performance; specifically walk/run mechanics in this example.

During the 3-4 day period using the sensor device 101X, the real-time feedback/push notifications to the processing device 200, 202 (e.g., his phone and/or smart watch) provide him information regarding:
1) Sitting time
2) Sitting posture (and time in posture) i.e. crossed legged, slouching, leaning, etc.
3) ‘Activities of Daily Living’ i.e. walking (any aberration from normal walking such as carrying bags to/from practice, bus, plane, etc.); ‘swagger’; standing duration (one-legged, leaning, etc.); relaxing/recovery time i.e. reclining or supine postures & asymmetries.
4) Training period
   a. Time on feet
   b. Walk vs. Run time (‘signatures’ for each)
   c. Standing behavior
   d. Sitting time/behavior
   e. Warm-up/cool-down tracking/behavior
5) Recommendations/Interventions: The player gets practical alternatives for postural mechanics (i.e. switch (or un-cross) crossed legs, switch shoulder carrying bag) to exercises to implement when he checks into hotel room after travel day (i.e. low back exercises; hip strengthening movements, etc.) relevant to postural behaviors he's been participating in that day as well as learned behaviors/trends/patterns that the monitoring and analysis system is picking up day-after-day. [This may be provided, for example, on a processing device 200, 202 in video, picture and short narrative form and may be part of an expanded educational and interventional service offering.]

Back-end Tracking/Shared data: Designated individuals (e.g., coaches, trainers, medical personnel, management, etc.) will be provided graphs & ‘pattern’ recognition reports at end of every (e.g., 24 hrs.) cycle, possibly through device 122 shown in FIGS. 1A and 1B. Summary and ‘key patterns’ will be highlighted that impact goals/priorities for them. For example, each participating party is given input options to screen data for relevant information only (i.e. physical therapist wants to know about asymmetrical postures and movement patterns relevant to past/pending injuries whereas a trainer wants to know about ‘time on feet’, recovery time; coaches/management want performance variables such as speed, footwork, work-rate beginning vs. end of practices, etc.). Each party may get different reports depending on their input specifications.
1. Recommendations/Interventions provided on processing device 200, 202: different recommendations/interventions may be suggested appropriate for the party involved. This will merely support their professional decision making/discretion. For example, the physical therapist may get scientifically published clinical guidelines for management of chronic low back pain through device 124; the team manager may get data on device 122 how/why to foster better equipment, seating arrangements for athlete, trainer my get data/suggestions for recovery options specific to that athlete based on data; the strength coach and physical therapist get examination and treatment recommendations for altered walk/run gait characteristics being identified.

Manual user input options (all of which may impact conclusions by the processing device 200):
1. The player sustains a minor sprain of his left ankle and he knows to manually input that information on the processing device 200, 202 so that the processing device 200, 202 can factor in both predicted walk/run gait abnormalities as well as anticipate compensations and re-calibrate recommendations to him.
2. The player doesn't sleep well one night so he manually reports feeling “tired”, “lethargic”, “weak”, etc. which gets factored in to the determinations and/or recommendations that may be made. Other subjective input can also be added by the player.

### B) Physical Therapy or Medical Patient

Information may go to physical therapists and/or other designated medical providers (available through device 123-124, for example) to establish a need for treatment plan changes, earlier appointment settings, documentation and background data prior to patients' next visit.
1) For example, if a diagnosis is previously established/known then the user feedback and intervention/treatment can be customized for the user.
2) There may be a backend option for the provider to make changes to feedback and intervention as a condition goes through phases.
3) The data may be stored in electronic form alongside the user's medical records as desired
4) The data may be shared with the user's Employer, Insurance company, or other relevant 3rd party (e.g., through devices 120-125).
5) Emphasis on data analysis may be tailored to see the ‘patterns’ of daily living that are feeding/contributing to the pain and dysfunction being treated. Real-time feedback to the user would be critical in order to change postural and movement behaviors in real time, or at least on a time scale shorter than a follow-up appointment.

### Case Example

A physical therapy patient with right hip pain has been referred to physical therapy by an orthopedic physician. MRI/x-rays indicate there may be a small labral tear in the hip and surgery is a consideration. However, the patient and physician would like to see if physical therapy can help avoid the cost and risk of surgery. The patient reports that their hip hurts throughout day, but especially when walking and standing for more than 15 minutes at a time. Additionally, the lower back has been cleared for being responsible for contributing to the symptoms. The patient states that the hip pain is at its worst when he transitions from sitting into walking and then eases as he walks, but then worsens the longer he walks or stands. The physical therapist finds hip mobility deficits and is able to reproduce symptoms with manual joint mobilization. Weakness is further identified to the gluteus medius/maximus and intra-abdominal core muscles. Tightness has also been noted to the hip adductors. Observational walking gait appears to have slight antalgic (pain avoidance) patterns with decreased stance time on right leg. The physical therapist desires to know what the daily ‘behaviors’ are of the patient so she prescribes the user wear the sensor device (e.g., 101X or 101Y of FIGS. 1A and 1B, respectively) of embodiments described above for 72 hours during waking hours.

During and after the 72 hours of using the sensor device 101X and/or 101Y, data are collected regarding the patient's sitting behaviors, duration, tendencies as well as walking/running behavior before and after sitting. Standing behaviors are also correlated with the activities of daily living (ADL's). The physical therapist's compensation strategies (i.e. what is suggested to alleviate the pain when standing/walking for more than 15 min.) such as, for example, lie down, sit; shift weight to the other side, etc. Based on the findings of the data, the physical therapist can tailor the patient education and coaching, the exercise program and manual therapy treatments around relevant ADL's of the patient. In other words, the physical therapist care can be more customized and provoking behaviors during the day, and the activities the patient participates in that worsen the condition can be identified using the data collected by the sensor(s) 101X and/or 101Y and analyzed using the processing device(s) 200, 202.

Tracking according to the embodiments described above would be used periodically throughout various phases during the course of one patient's treatments and with most patients on that physical therapist's schedule. The data collected by the sensors 101X and/or 101Y may be used, for example, for patient education and behavioral changes, as well as for documentation for physician and insurance companies who need to make decisions about whether or not patients are improving and whether coverage should continue to be paid or whether the physical therapist should move on to suggest surgery. This could provide significant healthcare cost savings. Patients may also use the sensor devices 101X and/or 101Y for ongoing monitoring after physical therapy has completed. Telehealth applications post-discharge may be provided to minimize changes of recurrence and return to the healthcare system (cost savings) by providing early identification of patho-mechanical/poor movement behaviors using the system described in embodiments above.

### C) Amateur/Recreational Athlete

Many of the over-all features and experience may be similar to those described in A) and B) above with additional support options. For example, a zip code referral may be used to provide referral recommendation based on postures, characteristic movements, and/or the correlation between the two for the user.

### D) Corporate Wellness

Many of the over-all features and experience may be similar to those described above. For example, the data may be shared with the employer using device 120 and/or the insurance company using device 123. Furthermore, push-notifications may be delivered to the user through the processing device (e.g., 200 in FIG. 1 or 202 in FIG. 2) to do chair exercises alongside streaming video(s) or demonstrative photo(s). The system may also be used for early identification, self-care options, and guidance.

### E) Senior Fall Risk/Prevention

Many of the over-all features and experience may be similar to those described above. Additionally, alert options for family, friends, and medical providers may be provided in the event certain characteristic movements (e.g., fall, no-motion tracking, etc.) are identified. Additionally, pattern recognition for early detection of disability and balance/gait changes may be determined.

### VII. Concluding Overview

Significant correlations may exist between the quality of a fundamental movement (such as running, jumping, etc.) performed by a person and the quality and/or duration of a body position (such as standing, sitting, lying down, etc.) held by the person shortly prior to that movement. It may in fact be useful to think in terms of all movement being in part the cumulative effect of static postural tendencies.

In some embodiments described herein, identification of a poor quality movement, such as walking with a limp, or ankle turning while running, may be correlated, using an AI system as described above, to a particular posture in their body position, such as leaning sideways while standing dominantly on one leg, slouching while sitting, lying down for an extended period in an asymmetrically dominant position, etc. Moreover, such a correlation may be made if the person adopts the particular posture shortly before the movement is carried out or if the particular posture is repeatedly over a long period of time (e.g., the body may start to “mal-adapt” in ways that alter the mechanics of movement).

Certain postures and body positions may even have a positive impact on subsequent movements.

Embodiments described herein are directed towards establishing whether such correlations exist for an individual user, so that, for example, the user may be guided to adopt better postures, or to change body positions more frequently, before carrying out particular movements. The data required to make such determinations are gathered with the aid of wearable inertial (IMU) sensor devices, causing minimal inconvenience and discomfort to the user. Many other uses for the information gleaned on body position, posture, and movement quality may be readily envisaged, including guidance for the user's clinical team, or a medical insurance provider, or the user's employer, if so authorized by the user.

Various simplified cases have been described above, but more complex systems may be envisaged that build on the same operating principles. As sensors, processors, and AI agents are improved in the future, more complex patterns of body position postures and movement characteristics may be identified, and correlations determined that involve, for example, foot scuffing, pelvic dropping, and behavior changes over time, etc. These patterns may then be added to the list of patterns that the service will detect and generate alerts from. It is possible that correlations may be found with defining individual characteristics (such as: height, weight, age, etc.), helping to identify people that are at higher risks for specific patterns of behaviors.

In conclusion, embodiments described herein provide various benefits. More specifically, embodiments allow for the convenient gathering and analysis of data indicative of user position, posture and movement quality, and correlations therebetween. Such data may be useful for many purposes, including clinical decision making, bio-feedback, insurance documentation, identifying injury risk, and identifying patterns/habits that limit movement performance and functional outcomes. Embodiments will allow improvements as well as declines in posture and movement to be tracked over time, in some cases over months or even years, and may provide new insights into fall risk, metabolic decline, and other conditions.

Embodiments may be implemented by using a non-transitory storage medium storing instructions executable by one or more processors to facilitate data entry by carrying out any of the methods described herein.

The above-described embodiments should be considered as examples, rather than as limiting the scope of the invention. Various modifications of the above-described embodiments will become apparent to those skilled in the art from the foregoing description and accompanying drawings. Accordingly, the present invention is to be limited solely by the scope of the following claims.

Additionally, references herein to “embodiment” means that a particular feature, structure, or characteristic described in connection with the embodiment can be included in at least one example embodiment of an invention. The appearances of this phrase in various places in the specification are not necessarily all referring to the same embodiment, nor are separate or alternative embodiments mutually exclusive of other embodiments. As such, the embodiments described herein, explicitly and implicitly understood by one skilled in the art, can be combined with other embodiments.

1. A method comprising:
receiving, by a first processing device, first data sent over a wireless communication channel from a first inertial sensor positioned on a user, wherein the first data are generated when the user is in a body position;
analyzing, by the first processing device, the first data to identify a posture of the body position;
receiving, by the first processing device, second data sent over a wireless communication channel from a second inertial sensor positioned on the user, wherein the second data are generated when the user performs a fundamental movement;
analyzing, by the first processing device, the second data to identify a distinguishing characteristic of the fundamental movement;
determining, by the first processing device, whether a correlation exists between the posture of the body position and the distinguishing characteristic of the fundamental movement; and
if a correlation between the posture of the body position and the distinguishing characteristic of the fundamental movement is determined to exist, displaying, on a second processing device worn by or positioned in close proximity to the user, an indication of the correlation.

2. The method of claim 1, wherein analyzing the first data comprises comparing the data to a first pattern, representative of the posture, to find a match, within a first tolerance, between a portion of the first data and the first pattern.

3. The method of claim 2, wherein the first pattern is established from data previously gathered from one or more subjects.

4. The method of claim 2, wherein the first pattern is established from data previously gathered from the user.

5. The method of claim 1, wherein analyzing the second data comprises comparing the second data to a second pattern, representative of the distinguishing characteristic of the fundamental movement, to find a match, within a second tolerance, between a portion of the second data and the second pattern.

6. The method of claim 1, wherein the determining, by the first processing device, whether a correlation exists between the posture of the body position and the distinguishing characteristic of the fundamental movement comprises:
determining a first time at which the user is in the posture of the body position;
determining a second time at which the user performs the distinguishing characteristic of the fundamental movement; and
evaluating the time interval between the first time and the second time as a possible first indicator of the correlation.

7. The method of claim 1, wherein the determining, by the first processing device, whether a correlation exists between the posture of the body position and the distinguishing characteristic of the fundamental movement comprises:
determining a first duration time for which the user remains in the posture of the firs body position; and
evaluating the first duration time as a possible second indicator of the correlation.

8. The method of claim 1, wherein the determining, by the first processing device, whether a correlation exists between the posture of the body position and the distinguishing characteristic of the fundamental movement comprises:
determining a second duration time for which the user remains performing the distinguishing characteristic in the fundamental movement; and
evaluating the second duration time as a possible third indicator of the correlation, and as a measure of long term significance of the correlation.

9. The method of claim 1, wherein the first inertial sensor is an IMU sensor placed on or near the center of mass of a user.

10. The method of claim 1, wherein the first processing device and the second processing device are the same device, worn by or positioned in close proximity to the user.

11. The method of claim 1, further comprising, if the first processing device is different from the second processing device, transmitting a message from the first processing device to the second processing device to cause the indication of correlation to be displayed.

12. The method of claim 1, wherein the first data and the second data each comprise 3-axis IMU data and an associated timestamp.

13. The method of claim 1, further comprising:
if the first processing device determines that the posture is of poor quality, displaying a first alert on the second processing device to notify the user of the poor quality posture.

14. The method of claim 1, further comprising:
if the first processing device determines that the distinguishing characteristic of the fundamental movement is problematic, displaying a second alert on the second processing device to notify the user of the problematic distinguishing characteristic.

15. A non-transitory computer-readable medium containing instructions executable by one or more processors of a computer system to:
receive, by a first processing device, first data sent over a wireless communication channel from a first inertial sensor positioned on a user, wherein the first data are generated when the user is in a body position;
analyze, by the first processing device, the first data to identify a posture of the body position;
receive, by the first processing device, second data sent over the wireless communication channel from a second inertial sensor, wherein the second data are generated when the user performs a fundamental movement;
analyze, by the first processing device, the second data to identify a distinguishing characteristic of the fundamental movement;
determine, by the first processing device, whether a correlation exists between the posture of the body position and the distinguishing characteristic of the fundamental movement; and
if a correlation between the posture of the body position and the distinguishing characteristic of the fundamental movement is determined to exist, display, on a second processing device worn by or positioned in close proximity to the user, an indication of the correlation.

16. The non-transitory computer-readable medium of claim 15, wherein analyzing the first data includes comparing the first data to a pattern representative of the posture, to find a match, within a first tolerance, between a portion of the first data and the pattern.

17. The non-transitory computer-readable medium of claim 16, wherein the pattern is established from data previously gathered from one or more subjects.

18. A system comprising:
a first processing device; and
a second processing device;
wherein the first processing device is configured to:
receive first data sent over a wireless communication channel from a first inertial sensor positioned on a user, wherein the first data are generated when the user is in a body position;
analyze the first data to identify a posture of the body position;
receive second data sent over the wireless communication channel from a second inertial sensor positioned on the user, wherein the second data are generated when the user performs a fundamental movement;
analyze the second data to identify a distinguishing characteristic of the fundamental movement;
determine whether a correlation exists between the posture of the body position and the distinguishing characteristic of the fundamental movement; and
if a correlation between the posture of the body position and the distinguishing characteristic of the fundamental movement is determined to exist, cause the second processing device to display an indication of the correlation.

19. The system of claim 18, wherein analyzing the first data includes comparing the first data to a pattern representative of the posture, to find a match, within a first tolerance, between a portion of the first data and the pattern.

20. The system of claim 18, wherein the first processing device and the second processing device are the same device, worn by or positioned in close proximity to the user.
